(19) United States
(12) Patent Application Publication
(10) Pub. No.: US 2014/0207121 A1
(43) Pub. Date: Jul. 24, 2014

(54) DRAINAGE CATHETER SYSTEM WITH CUFF

(71) Applicant: KIMBERLY-CLARK WORLDWIDE, INC., Neenah, WI (US)

(72) Inventors: Kelly D. Arehart, Roswell, GA (US); Alison S. Bagwell, Alpharetta, GA (US); Emily A. Reichart, Atlanta, GA (US)

(73) Assignee: KIMBERLY-CLARK WORLDWIDE, INC., Neenah, WI (US)

(21) Appl. No.: 13/745,971

(22) Filed: Jan. 21, 2013

(51) Int. Cl. A61M 27/00

(52) U.S. Cl. CPC A61M 27/00
USPC 604/543

(57) ABSTRACT

There is provided a drainage catheter system. The system includes an elongated tube having a substantially consistent cross-sectional size between a proximal end and a distal end. The tube has side walls defining a drainage lumen and the distal end of the tube is desirably closed. The tube further includes a plurality of holes in at least a portion of the side wall of the tube. These holes are provided along a length of the tube to define a drainage section of the catheter. The tube may include a hydrophilic coating on at least a portion of the exterior of the tube. The distal end of the tube may be tapered. The system also includes a head which is deployed outside the human body. The head is located on a proximal end of the tube and includes an opening to the drainage lumen of the tube.

This application claims the benefit of priority from U.S. Provisional Application No. 61/590,057 filed on Jan. 24, 2012, the contents of which are incorporated herein by reference.

### FIELD OF THE INVENTION

This invention relates to apparatus or equipment for draining fluid from a body cavity and methods of using such apparatus or equipment to drain fluid from a body cavity. More specifically, the invention relates to equipment or apparatus used for paracentesis and methods of using such apparatus or equipment to perform paracentesis.

### BACKGROUND

Clogging is a known problem for tubes used in drainage delivery systems, bowel management, nutrition, and dialysis. For example, many patients that require paracentesis procedures require them on a continuous basis and an indwelling catheter is placed for repeated access. Currently, these catheters are placed utilizing a tunneling technique. The tunneled catheters contain a polyester cuff at the most proximal end, to aid in promoting tissue in growth and act as a mechanical obstacle to bacterial infiltration. This cuff has been proven to be necessary to aid in cutting back on infection of the tract. However; the cuff makes it difficult to remove the catheter. When a tunneled catheter is clogged, kinked, or if the position of the tube is not allowing for proper drainage, there is a need to replace the catheter. When the catheter is damaged, it is necessary to remove the catheter and insert a new one. Replacing the catheter could cause additional trauma to the patient and the risk of increasing the probability of infection.

Drainage catheters are commonly placed utilizing a tunneling procedure. Using fluoroscopic guidance, a guide wire introducer with needle is inserted through the abdominal wall at the desired insertion site. The needle is removed while the guide wire introducer is left in place. An initial incision is made through the guide wire insertion site. A second incision is made 5-8 cm from the initial incision. A tunneler/catheter assembly is passed subcutaneously from the second incision down to and out through the incision at the guide wire insertion site until the polyester cuff on the catheter lies about 1 cm inside the second incision. The insertion site is dilated; a peel away introducer sheath is threaded over the guide wire and advanced into the peritoneal space. The guide wire and dilator are removed as a unit and the peel-away introducer sheath is left in place. The fenestrated end of the catheter is advanced into the sheath until all the fenestrations are within the peritoneal cavity. This can be verified under fluoroscopy as fenestrations are located along the barium sulfate stripe. The peel-away sheath is removed and the incision is closed at the insertion site. The catheter is then typically sutured to the skin superior to the second incision. Variations to the tunneling procedure can be performed (retrograde, antegrade, over the wire). However; all procedures utilize two incisions and the tunneling technique.

Infection is the most common complication associated with tunneled catheters. The distal end of the catheter is placed into the peritoneal cavity. Infection into the peritoneal cavity could lead to peritonitis which can result in patient death. Two incisions increase the risk of infection at the exit site.

### SUMMARY

The present invention addresses the problems described above by providing a drainage catheter system. The system includes an elongated tube having a substantially consistent cross-sectional size between a proximal end and a distal end. The tube has side walls defining a drainage lumen and the distal end of the tube is desirably closed. The tube further includes a plurality of holes in at least a portion of the side wall of the tube. These holes are provided along a length of said tube to define a drainage section of the catheter. The tube may include a hydrophilic coating on at least a portion of the exterior of the tube. The distal end of the tube may be tapered

The system also includes a head which is deployed outside the human body. The head is located on a proximal end of the tube and includes an opening to the drainage lumen of the tube. The head may include a low-profile feature that provides a bend in the tube of about 90 degrees, the bend being at a portion of the tube outside the body.

Another component of the system is a retention element which is deployed within a lumen of the body and which is located on a portion of the tube. The retention element may be a pigtail configuration of the elongated tube or an inflatable balloon or combinations thereof.

The system includes an epidermal tissue in-growth cuff. The epidermal tissue in-growth cuff may include or incorporate an antimicrobial treatment. For example, the antimicrobial treatment may be a silver treatment.

The system further includes at least one pressure indicator. The pressure indicator may be an indicator assembly composed of a first indicator element secured to the tube; a second indicator element secured to the tube; and an indicator located on the tube between the first indicator element and the second indicator element. The first indicator element and the second indicator element are configured to maintain substantially the same position with respect to each other on the tube and the indicator is configured to signal a change in position with respect to either the first or the second indicator element. This change in position may be used to signal a change in the pressure of body fluid in a lumen of the body. Alternatively and/or additionally, pressure indicator is an indicator for signaling the pressure of a retention element in the form of an inflatable balloon. In yet another embodiment, the pressure indicator is an indicator for signaling the pressure of body fluid in a lumen of the body, the body fluid being in fluid communication with a portion of the elongated tube.

Other objects, advantages and applications of the present disclosure will be made clear by the following detailed description.

### BRIEF DESCRIPTION OF THE DRAWINGS

These and other features of this invention will be more readily understood from the following detailed description of the various aspects of the invention taken in conjunction with the accompanying drawings in which:

FIG. 1A (on left) has a retention element that is a pigtail configuration of the elongated tube.

FIG. 1B (on right) has a retention element that is an inflatable balloon on the elongated tube.

### DETAILED DESCRIPTION

Reference will now be made in detail to one or more embodiments, examples of which are illustrated in the drawings. It should be understood that features illustrated or described as part of one embodiment may be used with another embodiment to yield still a further embodiment. It is intended that the claims include these and other modifications and variations as coming within the scope and spirit of the disclosure.

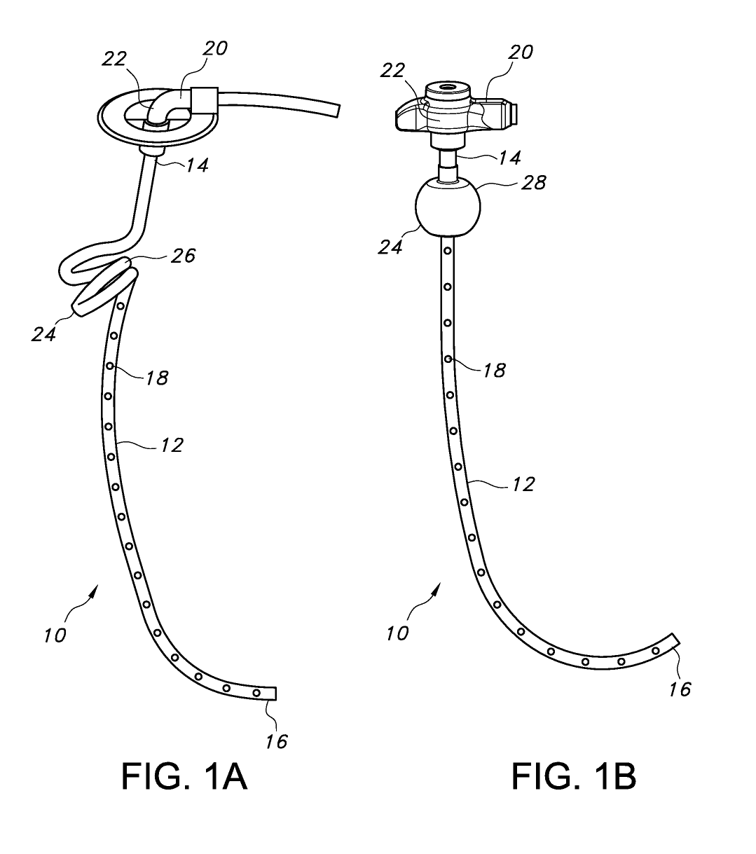

The present invention addresses the problems described above by providing a drainage catheter system. Referring now to FIGS. 1A and 1B of the drawings, the system 10 includes an elongated tube 12 having a substantially consistent cross-sectional size between a proximal end 14 and a distal end 16. The tube has side walls defining a drainage lumen and the distal end of the tube is desirably closed. The tube further includes a plurality of holes 18 in at least a portion of the side wall of the tube. These holes are provided along a length of said tube to define a drainage section of the catheter. The tube may include a hydrophilic coating on at least a portion of the exterior of the tube. The distal end of the tube may be tapered

The system also includes a head 20 which is deployed outside the human body. The head is located on a proximal end of the tube and includes an opening to the drainage lumen of the tube. The head may include a low-profile feature that provides a bend in the tube of about 90 degrees, the bend 22 being at a portion of the tube outside the body.

Another component of the system is a retention element 24 which is deployed within a lumen of the body and which is located on a portion of the tube. The retention element may be a pigtail configuration 26 of the elongated tube or an inflatable balloon 28 or combinations thereof. Exemplary retention elements are illustrated in FIGS. 1A and 1B respectively.

The system includes an epidermal tissue in-growth cuff. Such tissue in-growth cuffs are conventional and exemplary cuffs are described at, for example, U.S. Pat. No. 7,811,257 for “Medical Device with Adjustable Epidermal Tissue Ingrowth Cuff” issued Oct. 12, 2010 to Saab, the entire contents of which are incorporated herein by reference. The epidermal tissue in-growth cuff may include or incorporate an antimicrobial treatment. For example, the antimicrobial treatment may be a silver treatment.

The system further includes at least one pressure indicator. The pressure indicator may be an indicator assembly composed of a first indicator element secured to the tube; a second indicator element secured to the tube; and an indicator located on the tube between the first indicator element and the second indicator element. The first indicator element and the second indicator element are configured to maintain substantially the same position with respect to each other on the tube and the indicator is configured to signal a change in position with respect to either the first or the second indicator element. This change in position may be used to signal a change in the pressure of body fluid in a lumen of the body. Alternatively and/or additionally, pressure indicator is an indicator for signaling the pressure of a retention element in the form of an inflatable balloon. In yet another embodiment, the pressure indicator is an indicator for signaling the pressure of body fluid in a lumen of the body, the body fluid being in fluid communication with a portion of the elongated tube.

While the present invention has been described in connection with certain preferred embodiments it is to be understood that the subject matter encompassed by way of the present invention is not to be limited to those specific embodiments. On the contrary, it is intended for the subject matter of the invention to include all alternatives, modifications and equivalents as can be included within the spirit and scope of the claims.

We claim:

1. A drainage catheter system comprising:
an elongated tube having a substantially consistent cross-sectional size between a proximal end and a distal end, the tube having side walls defining a drainage lumen, the distal end of the tube being closed, and a plurality of holes in at least a portion of the side wall of the tube, the holes provided along a length of said tube defining an drainage section of the catheter;
a head which is deployed outside the human body, the head located on a proximal end of the tube and defining an opening to the drainage lumen; and
a retention element which is deployed within a lumen of the body, the retention element located on a portion of the tube,
an epidermal tissue in-growth cuff, and
at least one pressure indicator.

2. The system of claim 1, wherein the retention element is a pigtail configuration of the elongated tube or an inflatable balloon or combination thereof.

3. The system of claim 1, wherein the epidermal tissue in-growth cuff includes an antimicrobial treatment.

4. The system of claim 3, wherein the antimicrobial treatment is a silver treatment.

5. The system of claim 1, wherein the pressure indicator is an indicator assembly comprising:
a first indicator element secured to the tube;
a second indicator element secured to the tube; and
an indicator located on the tube between the first indicator element and the second indicator element,
wherein the first indicator element and the second indicator element are configured to maintain substantially the same position with respect to each other on the tube and the indicator is configured to signal a change in position with respect to either the first or the second indicator element.

6. The system of claim 1, wherein the pressure indicator is an indicator for signaling the pressure of a retention element in the form of an inflatable balloon.

7. The system of claim 1, wherein the pressure indicator is an indicator for signaling the pressure of body fluid in a lumen of the body, the body fluid being in fluid communication with a portion of the elongated tube.

8. The system of claim 1, wherein the head includes a low-profile feature that provides a bend in the tube of about 90 degrees, the bend being at a portion of the tube outside the body.

9. The system of claim 1, wherein the tube includes a hydrophilic coating on at least a portion of the exterior of the tube.

10. The system of claim 1, wherein the distal end of the tube is tapered.
